(19) United States
(12) Patent Application Publication
(10) Pub. No.: US 2010/0136119 A1
(43) Pub. Date: Jun. 3, 2010

(54) CONTROLLED-RELEASE PREPARATION CONTAINING CILOSTAZOL AND PROCESS FOR THE PREPARATION THEREOF

(75) Inventor: Jin Woo Park, Gangseo-gu (KR)

Correspondence Address: LAW OFFICES OF KHALILIAN SIRA, LLC, 9100 PERSIMMON TREE ROAD, POTOMAC, MD, 20854, US

(73) Assignees: AMOREPACIFIC CORPORATION, Seoul (KR); PACIFIC PHARMACEUTICAL CO., LTD., Seoul (KR)

(21) Appl. No.: 12/527,024

(22) PCT Filed: Feb. 15, 2008
(86) PCT No.: PCT/KR08/00901

§ 371 Date: Aug. 13, 2009

(30) Foreign Application Priority Data
Feb. 16, 2007 (KR) 10-2007-0016352

(51) Int. Cl. A61K 9/14; A61K 31/4709; A61P 9/10

(52) U.S. Cl. 424/489; 514/312

(57) ABSTRACT

A controlled release preparation which comprises particles containing cilostazol or its pharmaceutically acceptable salt dispersed in a solubilizing agent and an erodible material encasing said particles which is capable of forming a hydrogel, can maintain a constant level of cilostazol in the blood through its slow release during its prolonged residence time in the stomach and intestines, thereby minimizing adverse effects caused by rapid release of the drug or solubilizing agent.

### FIELD OF THE INVENTION

The present invention relates to a controlled release preparation of cilostazol and a method for preparing said preparation.

### BACKGROUND OF THE INVENTION

Cilostazol is a representative intracellular cAMP PDE (cyclic AMP phosphodiesterase) inhibitor and has been known to play significant roles in the suppression of the blood coagulation, the promotion of the central blood circulation, anti-inflammation and anti-ulcer actions, depression of blood pressure, the prevention and treatment of asthma and cerebral infarction, and the improvement of the cerebral circulation, by suppressing platelet coagulation and dilating the arteries through the inhibition of PDE activity.

Cilostazol has a poor water-solublity (1 μg/ml or less), and it has been demonstrated that orally administered cilostazol is absorbed mainly in the upper gastrointestinal (GI) tract and its absorption decreases as it moves to the lower GI tract. Therefore, the currently available preparations of cilostazol are of the form of a rapid release tablet because an ordinary controlled release formulation of cilostazol limits the absorption time at the desired absorption site. However, such a rapid release formulation of cilostazol can induce a sudden elevation of the drug concentration in the blood when orally administered, which results in adverse effects such as headache, and the dosage thereof is inconvenient in that the rapid release formulation should be administered twice a day in an amount ranging from 50 to 100 mg in order to maintain its pharmacological activity at a constant level.

Accordingly, there have been numerous attempts to develop a sustained or controlled release formulation of cilostazol that is free from the above problems.

For example, International Patent Publication No. WO 97/48382 discloses a sustained release formulation of cilostazol of a multiple unit form comprising at least 2 mini tablets prepared using hydroxypropylmethylcellulose as the main matrix, and International Patent Publication No. WO 96/21448 discloses a sustained release formulation of a resin particle form having a particle size less than 2,000 μm in diameter, which comprises cilostazol and ethylene-vinyl alcohol copolymer. However, these sustained release formulations exhibit ineffective drug absorption since the poorly soluble cilostazol having restricted absorption sites is released too slowly.

In order to solve this problem, International Patent Publication No. WO 00/57881 and U.S. Patent Publication No. 2002/0058066 have suggested a formulation comprising an external layer and a core, the external layer slowly releasing the drug in the upper GI tract (the small intestine) and the core releasing the drug rapidly in the lower small intestine and the colon. In addition, International Patent Publication No. WO 2005/023225 has reported a sustained release cilostazol formulation obtained by dissolving cilostazol in an organic solvent, depositing cilostazol on the surface of a porous inert carrier with additives such as microcrystalline cellulose, lactose, mannitol and sodium crosskamellose to increase the cilostazol's solubility, and then mixing the resulting product and a sustained release polymer. However, these formulations have the problems that the process for the preparation is very complicated, and the required daily dosage of the formulation becomes too large for a patient to take the drug comfortably.

### SUMMARY OF THE INVENTION

Accordingly, it is an object of the present invention to provide an improved controlled release preparation containing cilostazol or a pharmaceutically acceptable salt thereof.

It is another object of the present invention to provide a method for preparing said preparation.

In accordance with one aspect of the present invention, there is provided a controlled release preparation of cilostazol which comprises:

(A) particles comprising cilostazol or its pharmaceutically acceptable salt uniformly dispersed in a solubilizing agent, and

(B) an erodible material for forming a hydrogel in which said particles are dispersed therein.

In accordance with another aspect of the present invention, there is provided a method for preparing said controlled release preparation of cilostazol, which comprises:

(1) mixing cilostazol or its pharmaceutically acceptable salt with a solubilizing agent, and subjecting the resulting mixture to granulation using a solid dispersing method to produce particles; and

(2) adding an erodible material for forming a hydrogel to the particles obtained in step (1), and subjecting the resulting mixture to granulation to produce granules having the particles encased therein.

### DETAILED DESCRIPTION OF THE INVENTION

The controlled release preparation of the present invention may comprise cilostazol or its pharmaceutically acceptable salt, a solubilizing agent, and an erodible material for forming a hydrogel in amounts ranging from 10 to 80% by weight, 0.1 to 50% by weight and 5 to 80% by weight, respectively, based on the total weight of the preparation.

Hereinafter, the preparing method and the components of the inventive controlled release preparation are described in detail as follows:

### <Step (1)> Preparation of Drug Particles

In step (1) in accordance with the present invention, the cilostazol or its pharmaceutically acceptable salt and the solubilizing agent are mixed, and the resulting mixture is subjected to a solid dispersing method, to obtain particles having the drug dispersed uniformly therein.

The solid dispersing method may be any one of conventional melting or solvent methods. In case of using the melting method, the solubilized drug granules may be prepared by mixing the cilostazol with the solubilizing agent, heating the mixture to a temperature at which the cilostazol or the solubilizing agent does not melt to induce molecular level-mixing of cilostazol and solubilizing agent, slowly cooling the mixture to form a solid cluster, and processing the solid cluster to obtain particles of a desired size. When a solvent method is employed, the surface of cilostazol-containing particles is modified by the solubilizing agent, which may be prepared by dissolving the cilostazol and the solubilizing agent in a co-solvent, and drying the solution, or mixing cilostazol with a solubilizing agent dissolved or dispersed in a solvent in a high-speed rotation mixer or a fluid bed granulator.

1. Active Ingredient (Cilostazol)

In the controlled release preparation of the present invention, cilostazol or a pharmaceutically acceptable salt thereof is used as an active ingredient. Cilostazol may be employed in an amount ranging from 10 to 80% by weight, preferably 30 to 50% by weight based on the total weight of the preparation. When the amount is less than 10% by weight, the size of the preparation containing the recommended daily dose of cilostazol of 200 mg becomes too bulky to be orally administered to a patient, and when it is more than 80% by weight, the desired controlled release of the drug cannot be achieved.

2. Solubilizing Agent

In order to effectively control the release rate of cilostazol which has a low solubility in water of 1 μg/ml or less, the cilostazol in combination with the solubilizing agent is subjected to a solid dispersing method, producing drug granules having enhanced wetting property.

The solubilizing agent may be at least one ingredient selected from the group consisting of polyvinylpyrrolidone, copovidone, polyethyleneglycol, hydroxyalkylcellulose, hydroxypropylmethylcellulose, poloxamer, polyvinylalcohol, cyclodextrin and a surfactant. The surfactant may include, but are not limited to, at least one ingredient selected from the group consisting of an anionic surfactant, a non-ionic surfactant, an amphoteric surfactant, and a mixture thereof, preferable among which are a poly(oxyethylene)sorbitan fatty acid ester, poly(oxyethylene)stearate, a poly(oxyethylene)alkylether, a polyglycolized glyceride, a poly(oxyethylene)castor oil, a sorbitan fatty acid ester, a poloxamer, a fatty acid salt, a bile salt, an alkylsulfate, a lecithin, mixed micelles of bile salt and lecithin, a sugar ester vitamin E(polyethylene glycol 1000)succinate (TPGS), sodium lauryl sulfate, and a mixture thereof.

The solubilizing agent may be employed in an amount ranging from 0.1 to 50% by weight, preferably 5 to 20% by weight based on the total weight of the preparation.

3. Solvent

In case of selecting the solvent method in the solid dispersing method, water or an organic solvent may be used in a suitable amount, the organic solvent being methanol, ethanol, isopropanol, acetone, chloroform, dichloromethane, or a mixture thereof.

### <Step (2)> Final Granulation

In step (2) in accordance with the present invention, the drug particles containing a solubilizing agent obtained in step (1) and an erodible material for forming a hydrogel are mixed, and the resulting mixture is granulated. The granulating process may be conducted by conventional granulation methods which involve a step of dry granulation, wet granulation, melt granulation, fluid bed granulation; direct compression; molding; and extrusion molding, and the preferred method of which is the fluid bed granulation, wet granulation, melt granulation, dry granulation, or a combination thereof.

The granules obtained in step (2) may be formulated in the form of a capsule or a tablet.

4. Erodible Material for Forming a Hydrogel

Once the erodible material for forming the hydrogel is in contact with an external fluid, it immediately forms a hydrogel layer on the surface of the inventive preparation. The formation of the hydrogel layer imparts an inventive preparation three distinct physical properties: the inventive preparation consisting of a core part, an intermediate layer and an outermost layer, the core is not affected by the penetration of the external fluid; the intermediate layer attains the form of a rubber-like polymer; and the outermost layer becomes exposed to the external fluid. The boundary between the core part and the intermediate layer is referred to as “swelling front,” the boundary between the intermediate and outermost layers, as “diffusion front,” and the boundary between the outermost layer and the external fluid, as “erosion front.”

When a preparation containing cilostazol or its pharmaceutically acceptable salt is prepared by the conventional method, the core and the intermediate layer are each larger in volume than that of the erodible layer, which results in discharging the drug in an insufficiently solublized state. In this regard, in the present invention using a solid dispersing method, the wetting property of the low solubility drug is markedly improved, and the inventive preparation creates a remarkable change in the erodible layer disposed between the diffusion and erosion fronts to allow the drug separated from the erodible layer to dissolve fast.

Representative examples of the erodible material for forming the hydrogel include polyethyleneoxide, hydroxyalkylcellulose, hydroxypropyl alkylcellulose, polyvinylalcohol, polyvinylpyrrolidone, sodium carboxymethyl cellulose, propylene glycol alginate, carbopol, sodium alginate, xanthan gum, locust bean gum, cellulose gum, gellan gum, tragacanth gum, karaya gum, gua gum, acasia gum, and a mixture thereof. As the polyethyleneoxide, one having a molecular weight of 1,000,000 to 7,000,000 is preferred, and hydroxyethylcellulose and hydroxypropylcellulose are particularly preferred among the hydroxyalkylcellulose. Hydroxypropyl methylcellulose is preferred among the hydroxypropyl alkylcellulose.

The erodible material for forming the hydrogel may have a viscosity of 50 to 15,000 centipoises (cps), preferably 50 to 4,000 cps, more preferably 50 to 400 cps at room temperature of 25° C. This viscosity may be properly controlled within the above range depending on the desired drug release rate.

The erodible material for forming the hydrogel may be used in an amount of 5 to 80 wt %, preferably 10 to 50 wt %, based on the total weight of the preparation.

The inventive preparation may further comprise a pharmaceutically acceptable additive such as a binding agent, a diluent, a swelling agent and a lubricant added to the mixture before performing step (1) or (2), or to the granules obtained after conducting step (2).

Further, the granules obtained in step (2) may be coated with a coating agent, a plasticizer, a coloring agent, an antioxidant, talc, titanic dioxide, a flavoring agent, or a mixture thereof, in order to impart an inventive preparation desirable properties in terms of, e.g., color, stability, controlled release, suppression of burst release, and masking taste.

The coating agent or the binding agent may be employed in the form of a solution in water or an organic solvent, and the organic solvent may be methanol, ethanol, isopropanol, acetone, chloroform, dichloromethane, or a mixture thereof.

5. Diluent

Examples of diluents are lactose, dextrin, mannitol, sorbitol, starch, microcrystalline cellulose, calcium hydrogen phosphate, anhydrous calcium hydrogen phosphate, calcium carbonate, sugars, and a mixture thereof.

6. Binding Agent

Examples of binding agents are polyvinylpyrrolidone, copovidone, gelatin, starch, sucrose, methylcellulose, ethylcellulose, hydroxyethylcellulose, hydroxypropylcellulose, hydroxypropylalkylcellulose, and a mixture thereof.

7. Swelling Agent

Examples of swelling agents are cross-linked polyvinylpyrrolidone, cross-linked sodium carboxymethylcellulose, cross-linked calcium carboxymethylcellulose, cross-linked carboxymethylcellulose, sodium starch glycolate, carboxymethyl starch, sodium carboxymethyl starch, potassium methacrylate-divinylbenzene copolymer, amylose, cross-linked amylose, a starch derivative, microcrystalline cellulose, a cellulose derivative, cyclodextrin, a dextrin derivative, and a mixture thereof.

8. Lubricant

Examples of lubricants are stearic acid, stearate, talc, corn starch, carnauba wax, light anhydrous silicic acid, magnesium silicate, synthetic aluminum silicate, hardened oil, white lead, titanium oxide, microcrystalline cellulose, macrogol 4000 and 6000, isopropyl myristate, calcium hydrogen phosphate, and a mixture thereof.

9. Coating Agent

Examples of coating agents include at least one ingredient selected from the group consisting of ethylcellulose, shellac, ammonio methacrylate copolymer, polyvinylacetate, polyvinylpyrrolidone, polyvinylalcohol, hydroxymethylcellulose, hydroxyethylcellulose, hydroxypropylcellulose, hydroxybutylcellulose, hydroxypentylcellulose, hydroxypropylmethylcellulose, hydroxypropylbutylcellulose, hydroxypropylpentylcellulose, hydroxyalkylcellulosephthalate, sodium celluloseacetatephthalate, celluloseacetylphthalate, celluloseetherphthalate, anionic copolymer of methacrylic acid and methyl or ethyl ester methacrylate, hydroxypropylmethylcellulose phthalate, hydroxypropylmethylcellulose acetylsuccinate, cellulose acetylphthalate and Opadry™ (Colorcon Co.), and exemplary ammonio methacrylate copolymers may include Eudragit RS™ or Eudragit RL™.

10. Plastisizer

Examples of plastisizers include at least one ingredient selected from the group consisting of castor oil, fatty acid, substituted triglyceride and glyceride, triethylcitrate, polyethyleneglycol having a molecular weight of 300 to 50,000 and a derivative thereof.

In accordance with the inventive method, solubilized drug granules comprising cilostazol and a solubilizing agent are formed by a solid dispersing method and allowed to be uniformly dispersed in an erodible material for forming a hydrogel, thereby increasing influx of an external fluid close drug particles and enhancing the wetting property of the drug under a micro-environment to achieve the convenient control of drug release and absorption rates. Accordingly, the controlled release preparation of the present invention has advantages in that it maintains a constant level of cilostazol in blood through a slow release while it resides in the stomach and intestines over a long period of time, thereby minimizing adverse effects caused by rapid release of the drug or solubilizing agent and enhancing the patient compliance.

The following Examples are intended to further illustrate the present invention without limiting its scope.

### Examples 1 to 9

### Preparation of Tablet Containing Cilostazol—(1)

Cilostazol was placed in a high-speed rotation mixer, and sodium lauryl sulfate and hydroxypropylcellulose-L dissolved in ethanol were slowly added to the cilostazol undergoing high-speed rotation to obtain particles. Subsequently, the particles thus obtained were mixed with hydroxypropylmethylcellulose, crosslinked sodium carboxymethylcellulose or crosslinked polyvinylpyrrolidone, lactose, microcrystalline cellulose and calcium hydrogen phosphate, and hydroxypropylcellulose-L dissolved in ethanol was further added to the mixture. The resulting mixture was washed and sieved through No. 14 mesh to obtain granules, which were dried, further filtered through No. 18 mesh, and a magnesium stearate lubricant was added thereto. Then, the resulting mixture was compressed to obtain a tablet. The amounts of the ingredients used are shown in Table 1.

TABLE 1

| Ingredients | | | | | | | | | | |
|---|---|---|---|---|---|---|---|---|---|---|
| (mg) | | Ex. 1 | Ex. 2 | Ex. 3 | Ex. 4 | Ex. 5 | Ex. 6 | Ex. 7 | Ex. 8 | Ex. 9 |
| Cilostazol | | 200 | 200 | 200 | 200 | 200 | 200 | 200 | 200 | 200 |
| Hydroxy- | 400 | 50 | 100 | 140 | — | — | — | — | — | — |
| propylmethyl- | 4,000 | — | — | — | 50 | 100 | 150 | — | — | — |
| cellulose(cps) | 15,000 | — | — | — | — | — | — | 50 | 100 | 150 |

| | | | | | | | | | |
|---|---|---|---|---|---|---|---|---|---|
| Crosslinked sodium | 12.5 | 25 | — | 25 | — | 12.5 | 12.5 | 25 | — |
| carboxymethylcellulose | | | | | | | | | |
| Crosslinked | 12.5 | 25 | — | 25 | — | 12.5 | 12.5 | 25 | — |
| polyvinylpyrrolidone | | | | | | | | | |
| Lactose | 175 | — | — | — | 150 | — | — | 100 | — |
| Microcrystalline | — | 100 | — | — | — | 75 | — | — | 100 |
| cellulose | | | | | | | | | |
| Calcium hydrogen | — | — | 100 | 150 | — | — | 175 | — | — |
| phosphate | | | | | | | | | |
| Sodium lauryl sulfate | 25 | 25 | 25 | 25 | 25 | 25 | 25 | 25 | 25 |
| Hydroxypropylcellulose-L | 20 | 20 | 20 | 20 | 20 | 20 | 20 | 20 | 20 |
| Magnesium stearate | 5 | 5 | 5 | 5 | 5 | 5 | 5 | 5 | 5 |
| Total weight (mg) | 500 | 500 | 500 | 500 | 500 | 500 | 500 | 500 | 500 |

### Test Example 1

### Dissolution Test—(1)

The tablets obtained in Examples 1 to 9 were each subjected to a drug dissolution test using the USP dissolution test equipment. The time-dependent change of the drug dissolution rate was determined by the Paddle method conducted at 50 rpm/900 ml using a solution containing 0.5% sodium lauryl sulfate and 0.71% sodium chloride. The results are shown in Table 2.

TABLE 2

| Time (hr) | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|
| 0 | 0.00 | 0.00 | 0.00 | 0.00 | 0.00 | 0.00 | 0.00 | 0.00 | 0.00 |
| 1 | 23.33 | 9.36 | 6.47 | 15.40 | 8.59 | 4.65 | 14.54 | 9.28 | 4.05 |
| 2 | 47.31 | 17.38 | 13.81 | 28.98 | 18.62 | 9.46 | 29.70 | 16.46 | 8.49 |
| 3 | 67.13 | 35.20 | 21.41 | 42.17 | 28.65 | 15.25 | 43.11 | 23.19 | 12.66 |
| 4 | 80.91 | 42.11 | 29.25 | 56.57 | 37.91 | 23.22 | 54.00 | 30.06 | 18.83 |
| 6 | 101.31 | 61.90 | 44.80 | 80.08 | 53.83 | 35.87 | 77.45 | 48.29 | 31.48 |
| 8 | — | 79.16 | 59.85 | 94.85 | 66.46 | 48.57 | 92.50 | 64.10 | 44.35 |
| 10 | — | 96.30 | 70.31 | 101.81 | 81.22 | 60.36 | 101.50 | 78.88 | 56.84 |
| 12 | — | 100.59 | 84.23 | — | 92.80 | 70.78 | — | 91.10 | 68.44 |
| 14 | — | — | 93.44 | — | 100.86 | 79.39 | — | 98.80 | 78.93 |
| 16 | — | — | 99.19 | — | — | 87.07 | — | 101.02 | 87.56 |

As can be seen from Table 2, the release rate is primarily affected by the amount of the hydroxypropylmethylcellulose used for forming the hydrogel; it becomes slow as the amount of the hydroxypropylmethylcellulose increases. Also, the results obtained for Examples 2, 5 and 8 show that the release rate is significantly affected by the viscosity of the material used for forming the hydrogel. Accordingly, the drug release period of the inventive cilostazol tablet can be adjusted to a desired time length in the range of 6 hrs leading to 16 hrs through the control of the amounts or viscosities of the materials used for forming the hydrogel. Further, it is understand that the time-dependent change in the in vitro release pattern follows zero order kinetics.

### Comparative Example 1

### Preparation of Tablet Containing Cilostazol—(2)

Cilostazol, sodium lauryl sulfate, microcrystalline cellulose and propyleneglycol alginate were mixed, and hydroxypropylcellulose-L dissolved in ethanol was further added to the mixture. The resulting mixture was washed and sieved through No. 14 mesh to obtain particles, which were dried, further filtered through No. 18 mesh, and a magnesium stearate lubricant was added thereto. Then, the resulting mixture was compressed to obtain a tablet.

### Example 10

### Preparation of Tablet Containing Cilostazol—(3)

Cilostazol and sodium lauryl sulfate dissolved in ethanol were mixed, and the mixture was dried to form a solid mass. The mass was milled and then sieved through No. 20 mesh, to obtain particles. Subsequently, the particles were mixed with microcrystalline cellulose and propyleneglycol alginate, and hydroxypropylcellulose-L dissolved in ethanol was further added to the mixture. The resulting mixture was washed and sieved through No. 14 mesh to obtain granules, which were dried, further filtered through No. 18 mesh, and a magnesium stearate lubricant was added thereto. Then, the resulting mixture was compressed to obtain a tablet. The amounts of the ingredients used are shown in Table 3.

TABLE 3

| Ingredients | | Ex. |
|---|---|---|
| (mg) | Comp. Ex. 1 | 10 |
| Cilostazol | 200 | 200 |
| Propyleneglycol alginate | 150 | 150 |
| Microcrystalline cellulose | 105 | 105 |
| Sodium lauryl sulfate | 25 | 25 |
| Hydroxypropylcellulose-L | 15 | 15 |
| Magnesium stearate | 5 | 5 |
| Total weight (mg) | 500 | 500 |

### Test Example 2

### Dissolution Test—(2)

The tablets obtained in Comparative Example 1 and Example 10 were each subjected to a drug dissolution test using the USP dissolution test equipment. The time-dependent change of the drug dissolution rate was determined by the Paddle method conducted at 50 rpm/900 ml using a solution containing 0.5% sodium lauryl sulfate. The results are shown in Table 4.

TABLE 4

| Time (hr) | Comp. Ex. 1 | Ex. 10 |
|---|---|---|
| 0 | 0.00 | 0.00 |
| 1 | 6.37 | 15.18 |
| 2 | 14.97 | 31.81 |
| 3 | 23.51 | 48.62 |
| 4 | 32.40 | 64.52 |
| 6 | 50.64 | 90.75 |
| 8 | 66.96 | 101.23 |
| 10 | 81.26 | — |
| 12 | 92.60 | — |
| 14 | 100.70 | — |

As can be seen from Table 4, after 8 hrs, the tablet of Example 10 exhibited the dissolution rate close to 100%, whereas that of Comparative Example 1 exhibited the relatively low dissolution rate of about 67%, which suggests that the release rate of the tablet of Comparative Example 1 can be governed by the dissolution rate of the used drug, not by an amount or specific property of the material for forming the hydrogel.

### Examples 11 to 19

### Preparation of Tablet Containing Cilostazol—(4)

Cilostazol was placed in a high-speed rotation mixer, and sodium lauryl sulfate and hydroxypropylcellulose-L dissolved in ethanol were slowly added to the cilostazol undergoing high-speed rotation to obtain particles. Subsequently, the particles thus obtained were mixed with hydroxypropylmethylcellulose and microcrystalline cellulose, and hydroxypropylcellulose-L dissolved in ethanol was further added to the mixture. The resulting mixture was washed and sieved through No. 14 mesh to obtain granules, which were dried, further filtered through No. 18 mesh, and light anhydrous silicic acid and magnesium stearate lubricants were added thereto. Then, the resulting mixture was compressed to obtain a tablet. The amounts of the ingredients used are shown in Table 5.

TABLE 5

| Ingredients | | Ex. | Ex. | Ex. | Ex. | Ex. | Ex. | Ex. | Ex. | Ex. |
|---|---|---|---|---|---|---|---|---|---|---|
| (mg) | | 11 | 12 | 13 | 14 | 15 | 16 | 17 | 18 | 19 |
| Cilostazol | | 200 | 200 | 200 | 200 | 200 | 200 | 200 | 200 | 200 |
| Hydroxy- | A*: 4,000 | — | — | — | — | 150 | 120 | — | — | — |
| propyl- | B*: 4,000 | — | — | 150 | 120 | — | — | 75 | 50 | — |
| methyl- | 100 | 150 | — | — | — | — | — | 75 | 100 | 75 |
| cellulose | 15,000 | — | 150 | — | — | — | — | — | — | 75 |
| (cps) | | | | | | | | | | |

| | | | | | | | | | |
|---|---|---|---|---|---|---|---|---|---|
| Microcrystalline | 102.5 | 115 | 102.5 | 42 | 102.5 | 42 | 102.5 | 102.5 | 102.5 |
| cellulose | | | | | | | | | |
| Sodium lauryl | 25 | 15 | 25 | 20 | 25 | 20 | 25 | 25 | 25 |
| Sulfate | | | | | | | | | |
| Hydroxypropyl- | 15 | 15 | 15 | 12 | 15 | 12 | 15 | 15 | 15 |
| cellulose-L | | | | | | | | | |
| Light anhydrous | 2.5 | — | 2.5 | 2 | 2.5 | 2 | 2.5 | 2.5 | 2.5 |
| silicic acid | | | | | | | | | |
| Magnesium | 5 | 5 | 5 | 4 | 5 | 4 | 5 | 5 | 5 |
| stearate | | | | | | | | | |
| Total weight | 500 | 500 | 500 | 400 | 500 | 400 | 500 | 500 | 500 |
| (mg) | | | | | | | | | |

*note: A - Hydroxypropylmethylcellulose 2910 B - Hydroxypropylmethylcellulose 2208

### Test Example 3

### Dissolution Test—(3)

The tablets obtained in Examples 11 to 19 were each subjected to a drug dissolution test using the USP dissolution test equipment. The time-dependent change of the drug dissolution rate was determined by the Paddle method conducted at 50 rpm/900 ml using a solution containing 0.5% sodium lauryl sulfate and 0.71% sodium chloride. The results are shown in Table 6.

TABLE 6

| Time | | | | | | | | | |
|---|---|---|---|---|---|---|---|---|---|
| (hr) | Ex. 11 | Ex. 12 | Ex. 13 | Ex. 14 | Ex. 15 | Ex. 16 | Ex. 17 | Ex. 18 | Ex. 19 |
| 0 | 0.00 | 0.00 | 0.00 | 0.00 | 0.00 | 0.00 | 0.00 | 0.00 | 0.00 |
| 1 | 8.05 | 2.99 | 2.73 | 1.90 | 2.41 | 2.40 | 3.50 | 3.80 | 3.22 |
| 2 | 17.39 | 6.54 | 6.79 | 4.59 | 5.53 | 5.20 | 8.21 | 8.73 | 8.07 |
| 3 | — | 14.67 | 10.98 | 7.45 | 8.80 | 9.00 | 18.98 | 20.22 | 18.54 |
| 4 | 36.86 | 23.30 | 14.98 | 10.38 | 12.20 | 12.88 | 30.16 | 32.29 | 29.27 |
| 6 | 55.01 | 32.61 | 22.71 | 16.25 | 19.01 | 19.78 | 41.28 | 43.92 | 39.93 |
| 8 | 70.83 | 40.52 | 30.31 | 22.24 | 25.72 | 25.79 | 52.03 | 55.10 | 50.31 |
| 10 | 84.41 | 48.61 | 37.85 | 28.48 | 32.41 | 31.76 | 62.33 | 65.24 | 60.14 |
| 12 | 94.69 | 64.01 | 44.94 | 35.03 | 39.23 | 36.85 | 80.47 | 83.77 | 77.98 |
| 16 | 102.72 | 77.93 | 57.87 | 48.58 | 51.72 | 47.84 | 93.61 | 97.23 | 91.03 |
| 24 | 103.80 | 90.26 | 80.36 | 73.21 | 75.34 | 69.34 | 102.45 | 104.74 | 99.67 |

As can be seen from Table 6, the tablets of Examples 11 through 19 exhibited the time-dependent change in the in vitro release pattern corresponding to zero order kinetics over 16 hr- to 24 hr-periods. In other words, it is confirmed from these results that, in accordance with the present invention, the preparation having an enhanced drug dissolution rate can be advantageously obtained using minimized amounts of a solubilizing agent and a material for forming a hydrogel, thereby inhibiting the increasing of the preparation size as well as the early collapse of the preparation in the body.

Further, the above results show that the co-use of at least two hydrogel-forming materials with different viscosity makes a convenient control in the release rate possible.

### Examples 20 and 21

### Preparation of Coated Tablet Containing Cilostazol—(5)

The tablets prepared in Examples 11 and 19 were each successively spray-coated in a pan coater with a mixture of Opadry™ (Colorcon Co.) and purified water in amounts shown in Table 7.

TABLE 7

| Ingredients (mg) | Ex. 20 | Ex. 21 |
|---|---|---|
| Opadry | 20 | 20 |
| Purified water* | 113.33 | 113.33 |

*removed during the preparing procedure

### Test Example 4

### Absorption Test

In order to determine the bioavailability of cilostazol contained in the controlled release preparation, the tablets obtained in Example 21 and Comparative Example 2 (Pletaal™ (cilostazol 100 mg, Otsuka Pharmaceutical Co., Ltd.)) were respectively administered to beagle dogs (Beijing Marshall Biotechnology Co. Ltd., male, 5.5-week old, 6.94˜8.88 kg). The tablet obtained in Comparative Example 2 was further administered thereto after 6 hrs. Blood samples were taken from the dogs at regular intervals after the administration, and the time-dependent change of the blood drug concentration was analyzed. Based on the analyzed results, the maximum blood concentration (Cₘₐₓ), the time to reach the maximum blood concentration (Tₘₐₓ) and the area under the plasma concentration curve (AUC) of each tablet were calculated. The results are shown in Table 8.

TABLE 8

| | Cₘₐₓ (μg/ml) | Tₘₐₓ (hr) | AUC (μg * hr/ml) |
|---|---|---|---|
| Comparative Ex. 2 | 0.60 ± 0.26 | 2.60 ± 1.34 | 4.76 ± 2.81 |
| (100 mg * 2 times at the | | | |
| interval of 6 hrs, n = 6) | | | |
| Example 21 | 0.79 ± 0.22 | 3.50 ± 0.55 | 5.11 ± 1.07 |
| (200 mg * 1 time, n = 6) | | | |

As shown in Table 8, the tablet of Example 21 which was administered once exhibited Cₘₐₓ and AUC values which were equivalent to those of the tablet of Comparative Example 2 which was administered twice at the interval of 6 hrs, which suggests that the inventive preparation exhibits a satisfactory sustained release pattern. This suggests that the inventive controlled release preparation can maintain a desired effective level of cilostazol in blood even when administered once a day, thereby enhancing the patient compliance.

As described above, the controlled release preparation of the present invention can maintain a constant level of cilostazol in the blood through its slow release during its prolonged residence time in the stomach and intestines, thereby minimizing adverse effects caused by rapid release of the drug or solubilizing agent and enhancing the patient compliance.

While the invention has been described with respect to the above specific embodiments, it should be recognized that various modifications and changes may be made to the invention by those skilled in the art which also fall within the scope of the invention as defined by the appended claims.

What is claimed is:

1. A controlled release preparation of cilostazol which comprises:
(A) particles comprising cilostazol or its pharmaceutically acceptable salt uniformly dispersed in a solubilizing agent, and
(B) an erodible material for forming a hydrogel in which said particles are dispersed therein.

2. The controlled release preparation of claim 1, which comprises the cilostazol or its pharmaceutically acceptable salt in an amount ranging from 10 to 80% by weight; the solubilizing agent in an amount ranging from 0.1 to 50% by weight; and the erodible material for forming a hydrogel in an amount ranging from 5 to 80% by weight, based on the total weight of the preparation.

3. The controlled release preparation of claim 1, wherein the solubilizing agent is selected from the group consisting of polyvinylpyrrolidone, copovidone, polyethyleneglycol, hydroxyalkylcellulose, hydroxypropylmethylcellulose, poloxamer, polyvinylalcohol, cyclodextrin, surfactant, and a mixture thereof.

4. The controlled release preparation of claim 3, wherein the surfactant is selected from the group consisting of poly(oxyethylene)sorbitan fatty acid ester, poly(oxyethylene)stearate, poly(oxyethylene)alkylether, polyglycolized glyceride, poly(oxyethylene)castor oil, sorbitan fatty acid ester, poloxamer, fatty acid salt, bile salt, alkylsulfate, lecithin, mixed micelles of bile salt and lecithin, sugar ester vitamin E(polyethylene glycol 1000)succinate (TPGS), sodium lauryl sulfate, and a mixture thereof.

5. The controlled release preparation of claim 1, wherein the erodible material for forming the hydrogel is selected from the group consisting of polyethyleneoxide, hydroxyalkylcellulose, hydroxypropyl alkylcellulose, polyvinylalcohol, polyvinylpyrrolidone, sodium carboxymethyl cellulose, propylene glycol alginate, carbopol, sodium alginate, xanthan gum, locust bean gum, cellulose gum, gellan gum, tragacanth gum, karaya gum, gua gum, acasia gum, and a mixture thereof.

6. The controlled release preparation of claim 1, which further comprises a pharmaceutically acceptable additive.

7. The controlled release preparation of claim 6, wherein the pharmaceutically acceptable additive is selected from the group consisting of a diluent, a binding agent, a swelling agent, a lubricant, and a mixture thereof.

8. The controlled release preparation of claim 7, wherein the diluent is selected from the group consisting of lactose, dextrin, mannitol, sorbitol, starch, microcrystalline cellulose, calcium hydrogen phosphate, anhydrous calcium hydrogen phosphate, calcium carbonate, sugars, and a mixture thereof.

9. The controlled release preparation of claim 7, wherein the binding agent is selected from the group consisting of polyvinylpyrrolidone, copovidone, gelatin, starch, sucrose, methylcellulose, ethylcellulose, hydroxyethylcellulose, hydroxypropylcellulose, hydroxypropylalkylcellulose, and a mixture thereof.

10. The controlled release preparation of claim 7, wherein the swelling agent is selected from the group consisting of cross-linked polyvinylpyrrolidone, cross-linked sodium carboxymethylcellulose, cross-linked calcium carboxymethylcellulose, cross-linked carboxymethylcellulose, sodium starch glycolate, carboxymethyl starch, sodium carboxymethyl starch, potassium methacrylate-divinylbenzene copolymer, amylose, cross-linked amylose, a starch derivative, microcrystalline cellulose, a cellulose derivative, cyclodextrin, a dextrin derivative, and a mixture thereof.

11. The controlled release preparation of claim 7, wherein the lubricant is selected from the group consisting of stearic acid, stearate, talc, corn starch, carnauba wax, light anhydrous silicic acid, magnesium silicate, synthetic aluminum silicate, hardened oil, white lead, titanium oxide, microcrystallinecellulose, macrogol 4000 and 6000, isopropyl myristate, calcium hydrogen phosphate, and a mixture thereof.

12. The controlled release preparation of claim 1, which further comprise a coating layer containing a coating agent.

13. The controlled release preparation of claim 12, wherein the coating agent is selected from the group consisting of ethylcellulose, shellac, ammonio methacrylate copolymer, polyvinylacetate, polyvinylpyrrolidone, polyvinylalcohol, hydroxymethylcellulose, hydroxyethylcellulose, hydroxypropylcellulose, hydroxybutylcellulose, hydroxypentylcellulose, hydroxypropylmethylcellulose, hydroxypropylbutylcellulose, hydroxypropylpentylcellulose, hydroxyalkylcellulosephthalate, sodium celluloseacetatephthalate, celluloseacetylphthalate, celluloseetherphthalate, anionic copolymer of methacrylic acid and methyl or ethyl ester methacrylate, hydroxypropylmethylcellulose phthalate, hydroxypropylmethylcellulose acetylsuccinate, cellulose acetylphthalate, Opadry, and a mixture thereof.

14. A method for preparing the controlled release preparation of cilostazol of claim 1, which comprises:
(1) mixing cilostazol or its pharmaceutically acceptable salt with a solubilizing agent, and subjecting the resulting mixture to granulation using a solid dispersing method to produce particles; and
(2) adding an erodible material for forming a hydrogel to the particles obtained in step (1), and subjecting the resulting mixture to granulation to produce granules having the particles encased therein.

15. The method of claim 14, wherein the cilostazol or its pharmaceutically acceptable salt is used in an amount ranging from 10 to 80% by weight based on the total weight of the preparation.

16. The method of claim 14, wherein the solubilizing agent is used in an amount ranging from 0.1 to 50% by weight based on the total weight of the preparation.

17. The method of claim 14, wherein the erodible material for forming the hydrogel is used in an amount ranging from 5 to 80% by weight based on the total weight of the preparation.

18. The method of claim 14, which further comprises adding a pharmaceutically acceptable additive to the mixture before performing step (1) or (2), or to the granules obtained in step (2).

19. The method of claim 18, wherein the pharmaceutically acceptable additive is selected from the group consisting of a diluent, a binding agent, a swelling agent, a lubricant, and a mixture thereof.

20. The method of claim 14, which further comprises coating the granules obtained in step (2) with a coating agent.
